(19) United States
(12) Patent Application Publication
(10) Pub. No.: US 2019/0201243 A1
(43) Pub. Date: Jul. 4, 2019

(54) Vented Sound Attenuation Earplug System

(71) Applicant: Microsonic, Inc., Ambridge, PA (US)

(72) Inventors: Monika Pugliano, Ambridge, PA (US); Ozden Uslu, Ambridge, PA (US)

(73) Assignee: Microsonic, Inc.

(21) Appl. No.: 16/294,913

(22) Filed: Mar. 6, 2019

Related U.S. Application Data
(63) Continuation-in-part of application No. 15/817,141, filed on Nov. 17, 2017.

(51) Int. Cl. A61F 11/08

(52) U.S. Cl. CPC A61F 11/08; A61F 2011/085

(57) ABSTRACT

An earplug device that is placed in the ear canal to attenuate sound frequencies within a selected frequency range. The earplug has a plug body with a first end, an opposite second end, and an exterior surface that extends from the first end to the second end. A narrow vent channel is formed in the exterior surface of the plug body to enable air pressure to equalize by enabling a small volume of air to pass the earplug in the ear canal. The vent channel also enables a low level of sound to pass the earplug, therein enabling a person wearing the earplugs to understand spoken communications.

### RELATED APPLICATIONS

This application is a continuation-in-part of co-pending U.S. patent application Ser. No. 15/817,141, filed Nov. 17, 2017.

### BACKGROUND OF THE INVENTION

### 1. Field of the Invention

In general, the present invention relates to sound attenuation devices that are inserted into the auditory canal of the ear. More particularly, the present invention is related to sound attenuation devices that provide both decibel filtering and frequency filtering.

### 2. Prior Art Description

In an attempt to reduce the level of perceived noise, people often obstruct the auditory canals of their ears. Individuals have placed materials, such as cotton or paper fibers, into their ears for centuries. Such material easily conforms to the shape of the auditory canal. However, such materials rarely form a complete seal. Accordingly, loud sounds can still be perceived at levels that could damage the ear. In order to reduce noise levels, more and more material is packed into the auditory canal in an attempt to create a seal. This causes discomfort and can eventually cause pain or hearing damage if the material is driven against the ear drum.

In modern times, more sophisticated ear plugs are available. Such ear plugs are typically either amorphous or fixed. Amorphous earplugs include soft materials, such as silicone or soft foam. These amorphous materials are sold in small volumes that are just large enough to obstruct the ear canal. Such amorphous materials are inserted into the auditory canal and seal the auditory canal. However, as an individual moves, talks, eats and otherwise moves their mandible, the shape of the auditory canal changes. As the shape of the auditory canal changes, the amorphous material becomes deformed and gaps appear around the material. The gaps create openings though which loud sounds can propagate.

Fixed earplugs are typically molded or are custom formed using materials that cure after formation. However, achieving a perfect seal with a fixed material is not always possible due to changes in the shape of the ear canal caused by mandibular movement. This problem has been partially solved by using custom molded earplugs that are molded to the anatomy of a user's ear. Still, changes caused by mandibular movement move the earplug in the ear canal. The set shape of the material cannot adapt to continuous movement of the auditory canal. The result is that the earplug loosens and gaps form through which loud sounds can propagate.

Fixed molded earplugs have both advantages and disadvantages. An advantage of fix molded earplugs is that the earplug can be engineered to attenuate only certain decibel levels and certain frequency ranges. Such precision earplugs are typically manufactured with an internal sound attenuating filter. Such prior art is exemplified by European Patent No. EP2055277 to Oberdanner and U.S. Pat. No. 5,832,094 to LeHer. There are two primary disadvantages of fixed mold earplugs. First, the need for an attenuation filter makes the earplug difficult and expensive to manufacture. Second, such earplugs have an opening in their structure that faces the eardrum. This opening can, and commonly does, become blocked with cerumen. Once this happens, the engineered characteristics of the earplug are compromised and the intended attenuation levels become distorted. As a result, the hearing protection device does not perform as intended and the user is likely to avoid using the device. Acoustic filters have also been known to become loose over time and fall into the ear canal which can cause a health hazard.

Fixed molded earplugs also have a tendency to produce an occlusion effect in the user. The occlusion effect is a sense of increased sound pressure level, particularly in the low frequencies, that occurs when the ear canal is completely occluded. This causes a person to become sensitive to self-generated sounds, such as chewing, swallowing, breathing, and the like. This sensitivity is due to accumulation of sound pressure levels within the ear canal, especially that occur in low frequencies. This causes a hollow or echoic perception to low frequency sounds and vibrations that can be distracting and/or discomforting.

A need therefore exists for an improved earplug system that can form a better seal within the auditory canal, that is low cost, is easy to manufacture, and does not produce an occlusion effect. This need is met by the present invention as described and claimed below.

### SUMMARY OF THE INVENTION

The present invention is an earplug device that is placed in the ear canal to attenuate sound frequencies within a selected frequency range. The earplug has a plug body. The plug body has a first end, an opposite second end, and an exterior surface that extends from the first end to the second end. The exterior surface can be shaped to fit an average ear or can be custom molded to the anatomy of a specific person.

A vent channel is formed into the exterior surface of the plug body. The vent channel is narrow and extends between the first end and the second end of the plug body. The vent channel enables the pressure within the ear canal behind the earplug to equalize with ambient air pressure. The vent channel also enables a low level of sound to pass the earplug, therein enabling a person wearing the earplugs to understand spoken communications.

An opening is formed in the plug body at the first end. The opening leads to an internal conduit within the plug body. The internal conduit terminates at a closed membrane wall proximate the second end of the plug body. The internal conduit and the membrane wall both act upon incoming acoustic signals to both lower volume and attenuate certain undesired frequency ranges.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is a side view of an exemplary embodiment of an earplug shown in conjunction with the auditory canal of an ear;

FIG. 2 is a cross-sectional view of the embodiment of the earplug of FIG. 1, shown engaged within the auditory canal;

FIG. 3 shows an enlarged cross-sectional view of the exemplary earplug of FIG. 1 viewed along section line 3-3; and

FIG. 4 shows an enlarged cross-sectional view of the exemplary earplug in conjunction with an incoming audio signal and a reflected signal.

### DETAILED DESCRIPTION OF THE DRAWINGS

Although the present invention earplug system is typically sold in pairs for protecting both the left ear and the right ear, only one earplug is herein illustrated and described. It will be understood that the second earplug for the full set would have a mirrored geometry and would be manufactured and utilized in the same manner. The illustrated embodiment is selected for simplicity of description and represents one of the best modes contemplated for the invention. The illustrated embodiment, however, is merely exemplary and should not be considered a limitation when interpreting the scope of the appended claims.

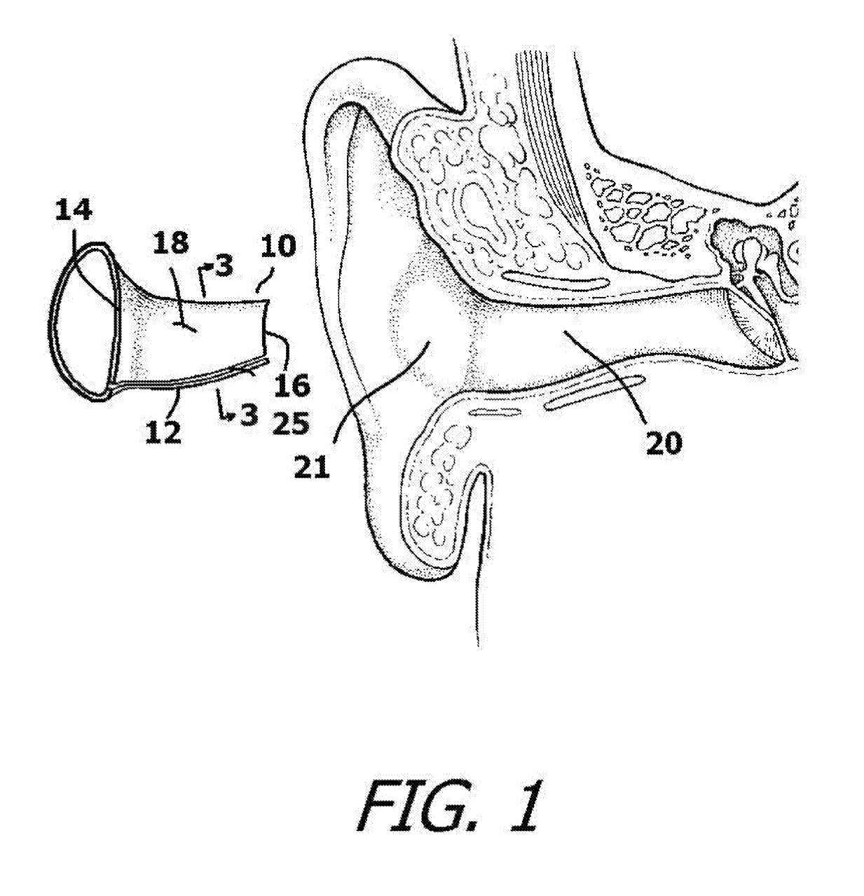

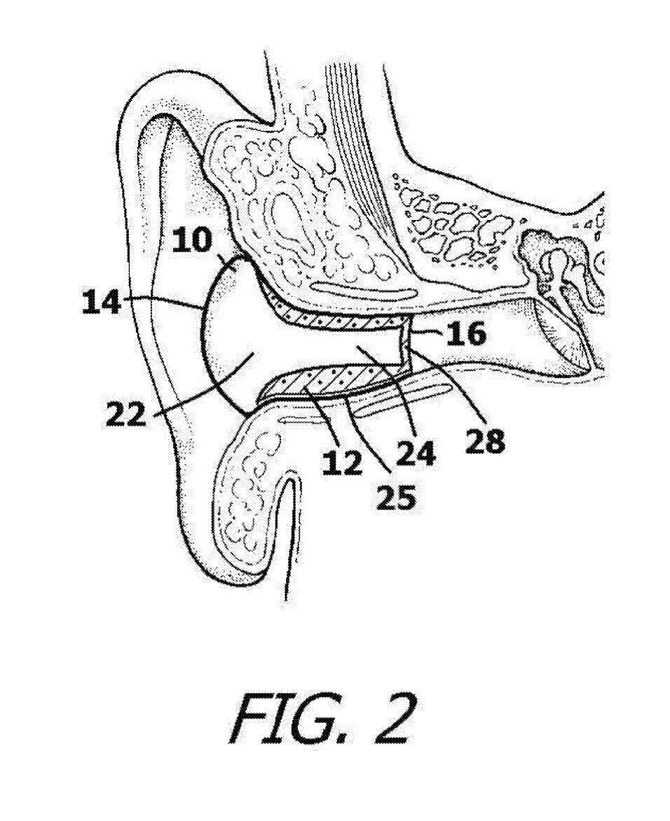

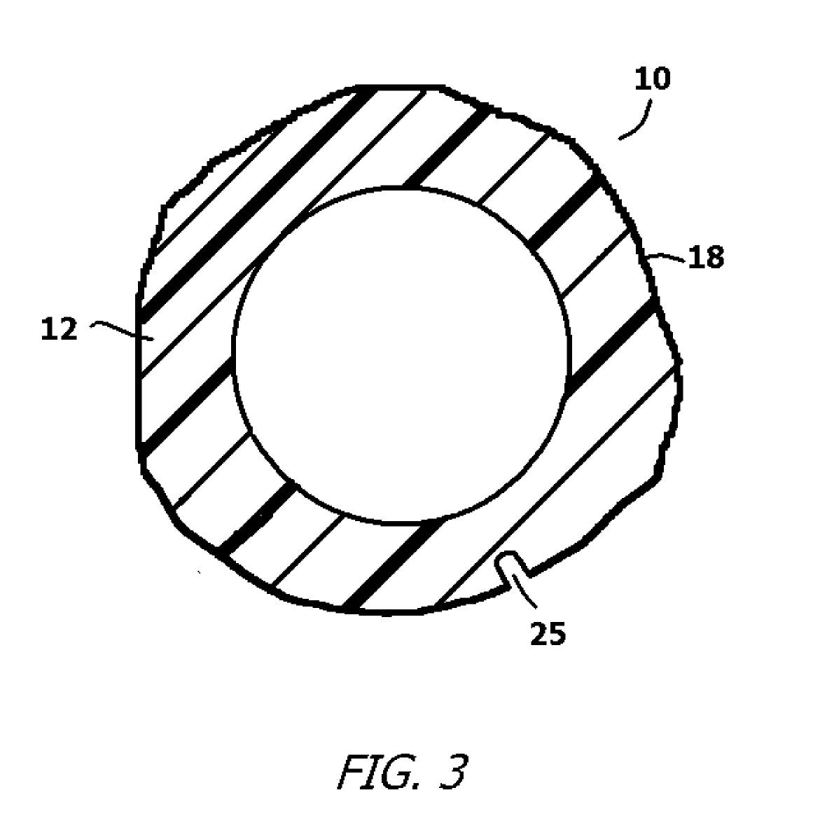

Referring to FIG. 1 in conjunction with FIG. 2 and FIG. 3, an earplug 10 is shown. The earplug 10 has a fitted plug body 12 that is generally horn shaped. That is, the plug body 12 has a first end 14 with a large diameter and a second end 16 with a small diameter, similar to the horn of trumpet. The plug body 12 has an exterior surface 18. The exterior surface 18 is anatomically shaped so that it matches the contours of the outer ear canal 20 and the concha bowl 21 that leads into the outer ear canal 20.

The exterior surface 18 of the plug body 12 matches the shape of the outer ear canal 20 with the exception of a vent channel 25. The vent channel 25 is a groove or depression formed into the exterior surface 18 of the plug body 12. The vent channel 25 extends between the first end 14 of the plug body 12 and the second end 16 of the plug body 12. The vent channel 25 does not become occluded when the earplug 10 is placed in the ear canal 20. This leaves a long narrow opening past the earplug 10 that enables air pressure within the ear canal 20 to gradually equalized with ambient pressure. This eliminates the occurrence of any occlusion effect that would otherwise occur from uneven pressures.

The vent channel 25 has a preferred cross-sectional area of between 0.125 mm² and 7.25 mm². Furthermore, the long narrow vent channel 25 is not straight. Rather, the vent channel 25 follows the complex curvature of the exterior surface 18 of the fitted plug body 12. The vent channel 25 starts at between inferior and medial tragal area and maintains a parallel line to inferior external contour until the first bend of the plug body, thereafter follows a straight line until the center of 2nd end of the plug body. The line described above is situated where the soft tissue is thinnest is followed to avoid filling of the external vent with soft tissue. The narrow size and complex shape of the vent channel 25 has the effect of attenuating loud noise, while enabling a small percentage of the softer sounds to pass. The earplug 10 will, therefore, attenuate loud sounds across the hearing spectrum, yet will still enable a person to hear spoken words. Likewise, the narrow complex shape of the vent channel 25 causes air pressure on either side of the earplug 10 to equalize slowly. This gradual venting prevents the popping and ringing of ears in barometrically dynamic environments, such as aircraft, elevators and the like.

Depending upon the degree of sound protection desired. The plug body 12 can be injection molded or 3D printed using a soft elastomeric material or soft curable polymer. The plug body 12 of the earplug 10 contacts the skin of the ear across the entire exterior surface 18 of the plug body 12 with the exception of the vent channel 25. As such, there is substantial material-to-ear contact across the exterior surface 18 of the earplug 10 from the first end 14 to the second end 16. The result is that although mandible movement of the user may create temporary gaps in the material-to-ear contact, the gaps are localized. The gaps never extend completely from the first end 14 to the second end 16. Accordingly, the integrity of the earplug 10 is not compromised and no sound energy can pass to the ear in an unintended manner.

Very little sound passes through the narrow vent channel 25. Rather, any sound energy that reaches the ear is primarily transferred through the earplug 10. The earplug 10 has a bell opening 22 at the first end 14 of the plug body 12. The bell opening 22 tapers down into an internal channel 24 that travels through the plug body 12 toward the second end 16. As will be later explained in more detail, the dimensions and length of the internal channel 24 are designed to produce an acoustic waveguide. The diameter of the internal channel 24 limits the amplitude of any acoustic signal entering the waveguide. The average diameter can be varied between 0.5 mm and 5 mm depending upon the size of the plug body 12 and the level of amplitude diminution desired. The distal end 26 of the internal channel 24, opposite the bell opening 22, is closed by a membrane wall 28. Accordingly, some of the acoustic energy that enters the internal channel 24 strikes the membrane wall 28 and is reflected back toward the bell opening 22. This reflected sound wave energy tends to interact with the incoming acoustical sound energy in an interference pattern that reduces the amplitude of the incoming sound energy. The result is a significant reduction in acoustical amplitude, which results in a corresponding reduction in sound volume.

The membrane wall 28 is engineered to create a specific level of amplitude and frequency filtering above and beyond that created by the dimensions of the internal channel 24. The thicker the membrane wall 28, the more sound energy is absorbed. Likewise, the thicker the membrane wall 28, the more acoustical energy is reflected back into the internal channel 24. The preferred thickness of the membrane wall 28 is between 0.2 mm and 5 mm depending upon the application. The greater the level of dangerous noise, the thicker the membrane wall 28 should be. Additionally, it is preferred that the membrane wall 28 be curved. The curved shape provides rigidity to the membrane wall 28 and prevents the membrane wall 28 from vibrating in sympathy with incoming acoustical energy. The result is that the membrane wall 28 will absorb acoustical energy rather than vibrate and will propagate that energy toward the second end 16 of the plug body 12.

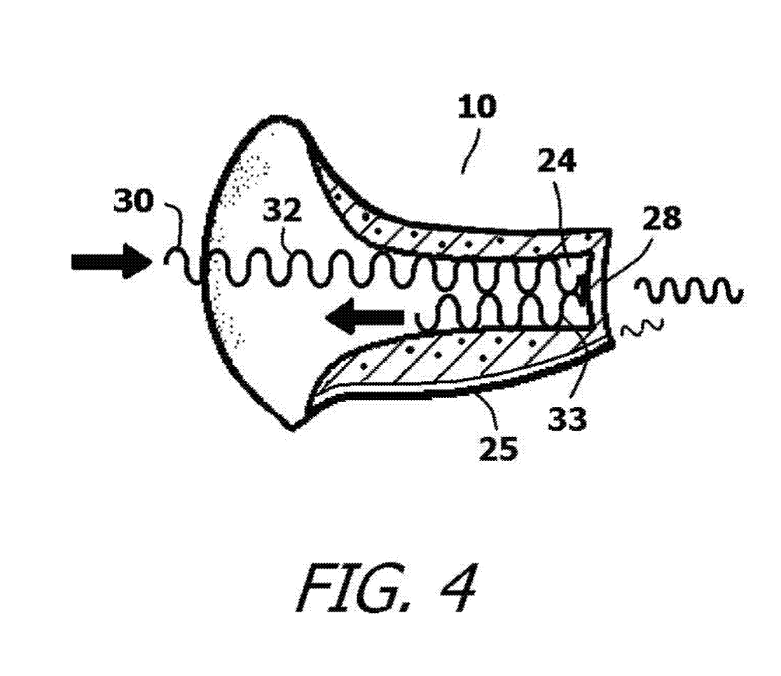

Referring to FIG. 4, it will be understood that both the structure of the internal channel 24 and the structure of the membrane wall 28 that terminates the internal channel 24, can be used to attenuate incoming audio signals 30. The degree of attenuation, however, can be controlled. Since the internal channel 24 and the membrane wall 28 together form an acoustic waveguide structure, it will be understood that these elements can be resonance-tuned to attenuate specific frequency ranges. This is accomplished by altering the dimensions of the internal channel 24 and the thickness of the membrane wall 28 so that incoming audio signals 30 within specific frequency ranges will be efficiently absorbed and/or reflected. This is achieved, in part, by having the incoming audio signals 30 impinge upon the membrane wall 28 at or near the largest amplitude of its waveform 32. This causes a reflected waveform 33 in an inverted phase to the incoming audio signals 30. The waveforms 32, 33 cancel each other and frequency filtering is accomplished. Accordingly, the length of the internal channel 24 should not be at or near a positive integer multiple of the undesired frequencies contained within the incoming audio signal 30.

It will also be understood that the dimensions of the vent channel 25, the internal channel 24 and the thickness of the membrane wall 28 can also be designed to allow certain frequencies within the incoming audio signals 30 to pass. The length of the internal channel 24 is controlled so that desirable frequencies within the incoming audio signals 30 travel through the internal channel 24 and impinge upon the membrane wall 28 near a null in the frequency waveform 34. This prevents the creation of an out-of-phase reflection and the incoming audio signals 30 can pass through the earplug 10 losing only the energy that is absorbed by the membrane wall 28. This can be accomplished by making the length of the internal chamber 24 a positive integer multiple of a desirable frequency that is to pass. In this manner, it will be understood that earplugs 10 can be custom designed to attenuate acoustic signals in certain frequency ranges but enable a person to hear other acoustic signals in desirable frequency ranges.

From the above, it will be understood that in order to have the internal channel 24 and the membrane wall 28 act as a tuned acoustic waveguide, the internal channel 24 and the membrane wall 28 must be manufactured to precise dimensions. This can be accomplished by taking an impression of the actual ear canal 20. The impression can then be scanned and used to create a mold, or can be used as a model for a 3D printer. In an alternative method of manufacture, the earplug 10 need not be custom manufactured. Rather, the earplug 10 can be mass produced using injection molding. The earplug 10 can be molded in a variety of sizes, such as small, medium and large. Using such a manufacturing technique, the earplug 10 can be mass produced for average people who have average ear anatomy. Such techniques will cause the earplug 10 to fit better on some individuals more so than on others. However, the earplugs 10 can be mass produced at low cost. Accordingly, the earplugs 10 can be marketed at low cost for disposable purposes, such as for use at music concerts or car races.

It will be understood that the embodiment of the present invention that is illustrated and described is merely exemplary and that a person skilled in the art can make many variations to that embodiment. All such embodiments are intended to be included within the scope of the present invention as defined by the claims.

What is claimed is:

1. An earplug device for placement in the ear canal, comprising:
a plug body having a first end, an opposite second end, and an exterior surface that extends from said first end to said second end; and
a vent channel formed along said exterior surface, wherein said vent channel extends between said first end and said second end.

2. The device according to claim 1, further including an opening at said first end that leads to an internal conduit within said plug body, wherein said internal conduit terminates at a closed membrane wall proximate said second end of said plug body.

3. The device according to claim 2, wherein said opening is largest at said first end and tapers into said internal conduit.

4. The device according to claim 1, wherein said vent channel has a cross-sectional area of between 0.125 square millimeter and 7.25 square millimeters.

5. The device according to claim 2, wherein said membrane wall has a thickness of between 0.2 mm and 5.0 mm.

6. The device according to claim 2, wherein said membrane wall has a wall surface that is curved.

7. The device according to claim 2, wherein said internal conduit has an average diameter of between 0.5 mm and 5.0 mm.

8. An earplug device that is placed in the ear canal to attenuate some sound frequencies within a selected frequency range, said device comprising:
a plug body having a first end, an opposite second end, an exterior surface and an internal conduit that is only accessible from said first end;
a vent channel formed into said exterior surface that extends from said first end of said plug body to said second end of said plug body.

9. The device according to claim 8, further including a solid membrane wall terminating said internal conduit within said plug body proximate said second end.

10. The device according to claim 9, further including an opening at said first end that tapers into said internal conduit.

11. The device according to claim 10, wherein said exterior surface is contoured to match an anatomical shape associated with the ear canal.

12. The device according to claim 9, wherein said membrane wall has a thickness of between 0.2 mm and 5.0 mm.

13. The device according to claim 9, wherein said membrane wall has a wall surface that is curved to stiffen said membrane wall.

14. The device according to claim 9, wherein said internal conduit has an average diameter of between 0.5 mm and 5.0 mm.

15. An earplug device that is placed in the ear canal to attenuate sound frequencies within a selected frequency range, said device comprising:
a plug body having a first end, an opposite second end, and an exterior surface custom molded to conform to said ear canal;
an internal conduit molded into said plug body, wherein said internal conduit is only accessible from said first end of said plug body; and
an external vent channel formed into said exterior surface that enables air to pass said plug body in said ear canal.

16. The device according to claim 15, further including a solid membrane wall that terminates said internal conduit within said plug body proximate said second end, wherein said internal conduit and said membrane wall cause attenuation in said selected frequency range of sound frequencies.

17. The device according to claim 16, further including an opening at said first end that tapers into said internal conduit.

18. The device according to claim 16, wherein said opening is largest at said first end and tapers into said internal conduit.

19. The device according to claim 15, wherein said vent channel has a cross-sectional profile area of between 0.125 millimeter and 7.25 square millimeters.

20. The device according to claim 16, wherein said membrane wall has a wall surface that is curved.
